(19) United States
(12) Patent Application Publication
(10) Pub. No.: US 2012/0040389 A1
(43) Pub. Date: Feb. 16, 2012

(54) METHOD FOR DEMONSTRATION OF A MOLECULAR EVENT IN A CELL BY MEANS OF FLUORESCENT MARKER PROTEINS

(75) Inventors: François Ichas, Pessac (FR); Francesca De Giorgi Ichas, Pessac (FR); Pier-Vincenzo Piazza, Bordeaux (FR); Jean Dessolin, Merignac (FR); Laura Schembri, Talence (FR); Flora Tomasello, Talence (FR); Lydia Lartigue, Bordeaux (FR)

(73) Assignee: Institut National de la Sante et de la Recherche Medicale (INSERM), Paris (FR)

(21) Appl. No.: 13/185,774

(22) Filed: Jul. 19, 2011

Related U.S. Application Data
(62) Division of application No. 10/563,451, filed on Jun. 5, 2006, now abandoned.

(30) Foreign Application Priority Data
Jul. 4, 2003 (FR) 03/08186

(51) Int. Cl. C12Q 1/37

(52) U.S. Cl. 435/23

(57) ABSTRACT

The invention relates to a method for demonstration of the occurrence of a molecular event, particularly in a cell, characterised by the detection of the ‘solubilisation’ of a fixed protein marker (or the fixing of a solubilised protein marker) which is a direct or indirect marker for the occurrence of the particular molecular event. Said protein marker is present in the cell before the above detection, the cell being subjected to a permeabilisation of the plasma membrane before detection, which liberates the solubilised protein into the extracellular medium, the presence of the marker protein thus being detected in the cell or the extra-cellular medium by any appropriate means, which permits the detection of whether the solubilisation, or the fixing have taken place and, hence, the corresponding molecular event.

This application is a divisional of U.S. application Ser. No. 10/563,451, filed on Jun. 5, 2006, which is a national phase entry under 35 U.S.C. §371 of International Application No. PCT/FR2004/001678 filed Jun. 30, 2004, published in WO2005/012913, which claims priority from French Application No. 03/08186 filed Jul. 4, 2003, the disclosures of which are incorporated herein by reference.

The present invention relates to a method for demonstration of a specific molecular event such as apoptosis in a living cell.

The demonstration of a specific molecular event such as apoptosis in a living cell usually involves a certain number of techniques such as the detection, by means of Western blot or immunofluorescence, of marker proteins that are implied in apoptosis phenomena, such as the Bax protein or activated caspases.

In the case of the caspases, for example, the methods for measuring the activation of these proteins in cells are essentially too long and too complex to carry out.

Thus, in the Western blot method, which is the usual method for observing caspase activation, antibodies are directed against caspase substrate proteins (PARP or caspase itself), and the appearance of molecular weight bands corresponding to the products of proteolysis are detected.

The technique is laborious, involves numerous preparatory steps, and enables the measurement of caspase activation only for populations of cells and not for individual cells.

The immunofluorescence technique uses antibodies which recognize, in a specific way, the active form of caspase and the technique can be used to detect it at the unicellular level on fixed cells. This technique makes it possible to detect individual cells but it is laborious and involves numerous preparatory steps.

Techniques that involve fluorescent chemical probes are also used. Several types exist commercially, and in general they consist of chemical products which have a strong affinity for the active sites of the protease and which emit a fluorescent signal once fixed to the caspase. These probes are capable of penetrating within cells and can make it possible to measure caspase activity by fluorescence microscopy and/or cytometry. However, their limitation is related to the loading protocol and to their cost which prevents their use for medium- or high-throughput tests.

Lastly, there are recombinant probes based on FRET technology which use genetically engineered probes that can be introduced into cells by transient or stable transfection. These probes consist of fluorescent proteins (which are GFP spectral mutants) and the detected signal results from the phenomenon of fluorescence energy transfer. The principle is that two GFP mutants bound together by a linker containing the cleavage sequence of the probe give a fluorescent transfer signal (emission from the acceptor molecule by excitation from the donor molecule due to the interaction between them). If caspase cuts the linker, the two proteins separate and the signal disappears.

These probes have been the subject of several recent publications but the difficulty is to detect the FRET signal (the need for using appropriate filters in microscopy and cytometry limits their use).

The method according to the present invention concern the detection of specific molecular events in a cell without using the aforementioned technologies while maintaining the ability to use microscopy and cytometry without requiring lengthy preparations or the use of particularly expensive reagents.

Indeed, as will be noted, the method according to the present invention requires no complicated preparation of the sample.

The cost is very low since the probe can be manufactured by the cell itself and measurement can be made on a single cell by way of fluorescence microscopy or by flow cytometry.

It is possible to create transgenic animals with this type of technology and it is also possible to transfer this test to microplates in order to create high-throughput systems.

The method according to the present invention to demonstrate the occurrence of a specific molecular event in a cell is characterized in that:
the “solubilization” of a “bound” marker protein (respectively the “binding” of a “solubilized” marker protein) that is a direct or indirect marker for the occurrence of the specific molecular event is detected,
said marker protein is present in the cell before the aforementioned detection,
the cell, before the detection, is subjected to a permeabilization of the plasma membrane which releases the solubilized protein into the extracellular medium,
the presence of the marker protein is then detected in the cell or in the extracellular medium by any appropriate means that makes it possible to determine if solubilization (respectively binding) has occurred, and thus the corresponding molecular event.

Usually the marker protein consists of a sensing component that undergoes solubilization (or binding) and of an indicator component that enables detection. As will be seen, this is often a fluorescent protein.

The sensing component can be a protein directly linked with the molecular event whose observation is sought, that is to say, that it is specifically its solubilization or its binding that constitutes the molecular event whose measurement is sought, or it can be a protein indirectly linked to the molecular event to be measured that is likely to undergo solubilization or binding following the induction of this molecular event; this is the case, for example, in proteolysis induced by caspases during apoptosis.

In any event, the sensing component protein must undergo solubilization, respectively binding, when the molecular event occurs.

In the present description, the “binding” of a protein means the subcellular anchoring, for example, to the membrane, to the nucleus, or to the inter-membrane mitochondrial space, etc., preferably to the membrane, or the subcellular compartmentalization of the aforesaid protein whereby the protein cannot diffuse into the extracellular medium during the permeabilization of the cell. In the same way, cellular “solubilization” of the protein indicates the presence of the marker protein in the form of a free protein in the cell cytosol, such that the protein can diffuse into the external medium during the permeabilization of the cell.

When selective permeabilization of the plasma membrane is carried out, if the marker protein is bound, that is to say, if it is anchored at the subcellular level, on a compartmentalized membrane in particular, it will remain bound within the cell and will not pass into the extracellular medium; on the contrary, if the protein is soluble, that is to say, present in the cytosol, it will migrate into the extracellular medium Observed under these conditions, cells in the first case will be marked and cells in the second case will not be marked.

The marking of the cell being preferably marking by fluorescence, it is then easy to detect marked and unmarked cells by fluorescence microscopy or by cytometry. This requires no preparation of the sample except for permeabilization.

It is theoretically possible to use other types of markers which would make it possible to distinguish the presence or, on the contrary, the absence of the marker protein in the cell, but it is certain given developments in fluorescence techniques that this is the technique which will be preferred.

Thus in a cell population expressing the marker homogeneously, selective permeabilization of the plasma membrane succeeds in the discrimination of cells in which the molecular event has taken place because the fluorescent signal will be specifically maintained (or respectively lost) in this cell subpopulation.

Analysis by flow cytometry makes it possible to evaluate quantitatively the percentage of the cell population in which the measured molecular event has taken place and provides a simple test to evaluate the activating and inhibiting effects of drugs that interact with the aforesaid molecular event.

The approach described here only requires a very limited experimental manipulation (no fractionation, no antibody fixation or incubation, no electrophoresis, no microscopy). The cumulative time for cell recovery, permeabilization, and analysis is less than 30 minutes (other techniques require several hours, even days).

It makes it possible to measure the parameter of interest within the cell without inducing artifacts related to fractionation (WB) or to fixation and the use of detergents in high amounts (immunofluorescence).

Lastly, this technique presents low experimental costs (no reagents, no antibodies).

The marker protein can be produced transiently in the cell by way of an expression vector of the aforesaid protein, but it is also possible to anticipate the constitutive expression of this marker protein in cell lines or transgenic animals that will thus be capable of becoming tools for the detection of the molecular event. In the case of apoptosis, for example, these tools will make it possible to test products having pro- or anti-apoptotic properties without having to carry out transfections by vectors appropriate to each test.

The technique that enables the expression of a marker protein is known in the prior art; the plasmids or vectors that may be used will depend, of course, on the cell, as well as the promoters and the various elements involved in the regulation of expression.

Similarly, the techniques that enable the constitutive expression of a marker protein are known; they involve introducing a stable or inducible marker protein expression system into one of the chromosomes of the cell by recombinant methods, for example.

In a preferred embodiment of the invention, the marker protein is comprised of a fusion protein that includes:
a sensing protein which itself undergoes a solubilization, respectively a binding, during the occurrence of the molecular event,
a fluorescent fragment, in particular a fluorescent protein: green fluorescent protein, red fluorescent protein, or any molecule which derives from it or which has similar properties of fluorescence.

In the examples, the proteins EGFP, DsRed2, HcRed, and copGFP were used, but other proteins can be used such as spectral mutants of the preceding proteins.

Generally, protein markers can be considered of two types. In the first, the sensing component is constituted by the protein element that undergoes solubilization or binding, this molecular event being what is sought to be measured directly. This is the case for example of Bax, which will be described more completely below. In the second type of marker protein, the sensing component can be a protein fragment which is solubilized or bound following the interaction with a protein that undergoes the molecular event that is sought to be measured. This is the case, for example, with caspase activation: the marker protein will be a fusion protein containing the protease cleavage site and, on either side of this cleavage site, a membrane anchoring site and a fluorescent protein. Thus, during protease activation, the component indicator, that is to say, the fluorescent protein, will be solubilized by cleavage.

In the latter case, the anchoring protein could be selected in particular among the known transmembrane domains; in particular it will be an exogenous transmembrane domain attached at the end of the fluorescent protein via a linker which will contain the protease cleavage site.

In the description and examples which follow, the transmembrane domain that has been tested is a short C-terminal domain present in class of proteins known as tail-anchored (TA) proteins. This transmembrane portion was conferred mitochondrial specificity by mutation but it is possible to use other protein anchoring cassettes in the membrane, for example the myristoylation or palmitoylation sequence N-terminal transmembrane domain, or addressed to other membranes (plasma membrane, membrane of the endothelial reticulum, Golgi for example), provided that the fluorescent part remains exposed in the cytosol.

The technique can be applied to proteins naturally possessing this property or to proteins artificially built for this purpose.

The present invention thus relates to a method for the demonstration of the occurrence of a specific molecular event, and this is accomplished by way of a marker protein.

As is illustrated in the examples which follow, the method according to the invention can be implemented by coupling the demonstration of the occurrence of the molecular event with the measurement of the cell cycle, in particular the measurement of the distribution of the cell population in the various phases of the cell cycle.

In particular, such a coupling can be performed when the molecular event to be detected is the Bax activation (see example 6a below), or the activation of a caspase such as caspase 3 (see example 6a).

But the present invention also relates to:
protein markers useful in the implementation of this method, that contain a sensing component which will undergo solubilization (binding) and an indicator component which will allow detection.

Preferably, this will be a fusion protein where the indicator component is a fluorescent protein, as that which has been mentioned previously.

According to one embodiment, a marker protein in conformity with the invention contains a sensing component whose sequence is coded by a nucleic acid that includes a sequence chosen among the sequences SEQ ID Nos. 1, 3, 5, 7, 9, 11, and 13.

According to another embodiment, a marker protein according to the invention contains a sensing component whose sequence includes a sequence chosen among the sequences SEQ ID Nos. 2, 4, 6, 8, 10, 12, and 14.

The invention also relates to:
vectors that express, in an appropriate cell environment, a marker protein,
transformed cells that express a marker protein in a stable or transient way, with the aforesaid cells capable of being tumor cells, preferably human tumor cells.

The invention relates to non-human transgenic animals in which at least one type of cells expresses a marker protein.

The invention also relates to a kit for implementation of the method according to one of the preceding embodiments, comprising:
transformed cells; and/or
a vector; and/or
a transgenic animal,

such as mentioned previously.

In addition, one aspect of the invention is a method to evaluate the activity of a candidate anti-cancer compound.

Such a method includes the implementation of a method for the demonstration of the occurrence of a specific molecular event in a transformed tumor cell, preferably of human origin, expressing a marker protein.

Within the framework of the invention, a “compound” is defined as being any type of molecule, whether biological, chemical, natural, recombinant, or synthetic. For example, a compound can be a nucleic acid (an oligonucleotide, for example), a protein, a fatty acid, an antibody, a polysaccharide, a steroid, a purine, a pyrimidine, an organic molecule, a chemical radical, etc. The term “compound” also covers fragments, derivatives, structural analogs, and combinations of the above.

A eukaryotic cell uses various strategies to appropriately activate signal transduction pathways in response to specific stimuli. In addition to transcriptional control (the active molecule is synthesized de novo) and post-translational control (the signal molecule already present undergoes a change that activates it, such as proteolysis, phosphorylation, or a protein-protein interaction), in certain cases the cell invokes a strategy of compartmentalization: the active molecule is already expressed in the cell, but it is trapped in a subcellular compartment where it cannot perform its function (for example, release of pro-apoptotic factors from the mitochondrial matrix, factor recruitment in the plasma membrane).

In certain cases, this type of strategy not only involves a change in intracellular distribution but also a change in solubility of the protein which can be in soluble cytosolic form in its inactive form and associated with the membrane in its active form, or vice versa (for example, pro-apoptotic proteins of the Bcl-2 family, transcription factors activated by the endoplasmic reticulum). This type of event can thus be demonstrated by microscopy and by flow cytometry by way of the construction of a fusion protein combined with a fluorescent protein (see example below: probe for measuring Bax activation).

Similarly, it is possible to apply this type of approach to the use of marker proteins which have artificially been given the property of changing phase with respect to the signal to be measured. In particular, recombinant marker proteins can be constructed for measuring the activity of intracellular proteases. In these marker proteins, the fluorescent protein is attached to an intracellular membrane by fusion with a transmembrane domain by means of a linker sequence which contains the cleavage site of the protease whose activity is sought to be measured. It is this type of approach that was adopted for the caspase 3 probe described in the examples.

The permeabilization step is not essential in theory. Indeed, it is possible by fluorescence microscopy, for example, to detect the presence of fluorescence in the cytosol, on the membrane, or within the compartments in which it is bound, however permeabilization of the cells allows an automation of the process and constitutes the preferred embodiment of this invention.

In order to enable salting-out of the fluorescent marker protein in soluble form in the extracellular medium, all suitable technologies known to those skilled in the art can be used. In particular, it will be preferable to use permeabilization by digitonin at concentrations in the range between 1 and 100 μM/ml, and preferably in the range between 5 to 50 μM/ml, but it is also possible to use other detergents such as saponins in very small amounts, for example from 0.5 to 10 μM/ml, preferably on the order of 1 μM, streptolysin O (20-500 ng/ml), or freeze-thaw cycles.

This allows detection to be made, for example by flow cytometry, which is obviously much more sensitive and automatable than other techniques, even if they too can be used, such as a microplate reader, a fluorescence microscope, or a confocal microscope, for example.

The methods according to the present invention are valuable for demonstrating a large number of molecular phenomena, in particular the study of anti- and pro-apoptotic properties of molecules intended to be used as medicines.

Apoptosis is a highly preserved and controlled process of cell death, consisting of a cascade of molecular events which lead the cell towards degradation and death (1). Abnormal apoptosis is the source of a number of cancers (lack of apoptosis) and is also implied in the pathogenesis of neurodegenerative processes (excess of apoptosis) (1).

The final phase of apoptosis is constituted by the degradation of cell structures, on the one hand by the effect of the activation of a specific class of cysteine proteases, caspases, and on the other hand by the activation of endonucleases which degrade nuclear chromatin (1).

A cell which undergoes the process of programmed cell death is characterized by many more or less specific morphological and biochemical signs. Certain somewhat specific morphological changes are easily detectable by simple observation using optical microscopy, such as cell condensation, the formation of plasma membrane blebs, and the appearance of permeability of the latter to propidium iodide. Some nuclear dyes (Hoechst, DAPI) that reveal the morphology of the nucleus make it possible to visualize the degradation/condensation of chromatin. This latter parameter is also detectable by electrophoresis of genomic material of a cell population by the appearance of the typical DNA ladder pattern, and at the unicellular level by microscopy and by flow cytometry thanks to the TUNEL principle, colorimetric or fluorescent marking that titrates the quantity of free ends of DNA produced by the action of apoptotic endonucleases. Among the other signs generally measured are included: caspase activation (Western blot, flow cytometry), proteolysis of activated caspase substrates (Western blot), and the exhibition of phosphatidylserine on the external layer of the plasma membrane (flow cytometry).

Upstream of the processes described above, there are various intracellular signal pathways which induce the cell to begin a process of self-destruction (the initiation of apoptosis): these apoptotic induction stimuli are diverse, and the corresponding intracellular signal cascades can vary according to the inductive stimulus and/or the cellular model.

Some of these early molecular events represent key pro-apoptotic steps, and the possibility of detecting them specifically with good sensitivity opens, for example, the possibility of screening for anti- or pro-apoptotic active compounds.

The relocalization of the Bax protein from the cytosol to the mitochondrial membrane is an early and generic event during the signaling of apoptosis (2). The relocalization of Bax, followed by homo-oligomerization, is the cause of the release of cytochrome c which results irrevocably in death of the cell by causing the activation of caspase 9, then that of caspase 3 (2). Bax is a globular cytosolic protein whose primary structure enables it to be classified in the Bcl-2 family. The work of Youle (2) largely contributed to the understanding of the role played by the various Bax domains in its relocalization and its redistribution, however its secondary structure was unknown until the study by Tjandra which allowed its elucidation by nuclear magnetic resonance (NMR) (2). The conformation of the C-terminal domain of Bax, comprised by the helix α9 containing 22 residues, proved of major importance. In the cytosolic soluble form of the protein, this helix rests in a hydrophobic cavity and relocalization depends on a conformational change of Bax that exposes the helix outside the hydrophobic pocket: the “exposed” C-terminal domain of Bax then possesses a tropism for the mitochondrial membrane. It is this change in conformation that can be demonstrated by the method according to the invention, as will be described in the examples.

Other characteristics and advantages of the present invention will appear in the reading of the following examples while referring to the appended figures in which:

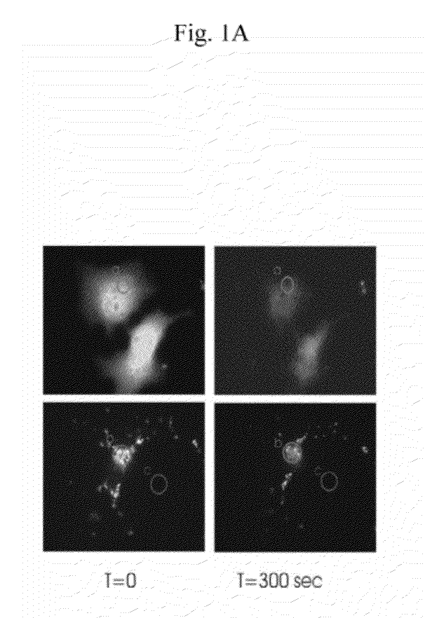

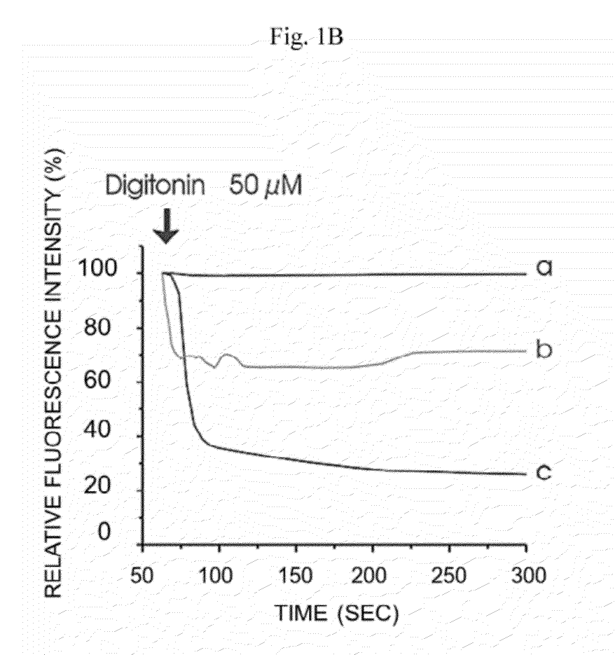

FIG. 1A-B represents fluorescence microscopy of a human cell clone (clone 10) obtained from a HeLa line that stably expresses the chimeric protein GFP-Bax at T=0 and T=300 s without permeabilization and with permeabilization by digitonin and the curves corresponding to points a, b and c;

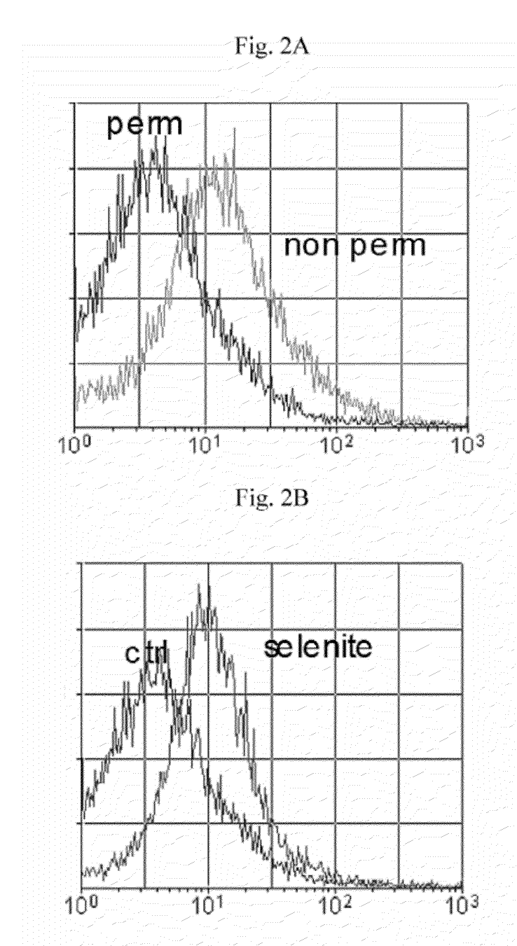

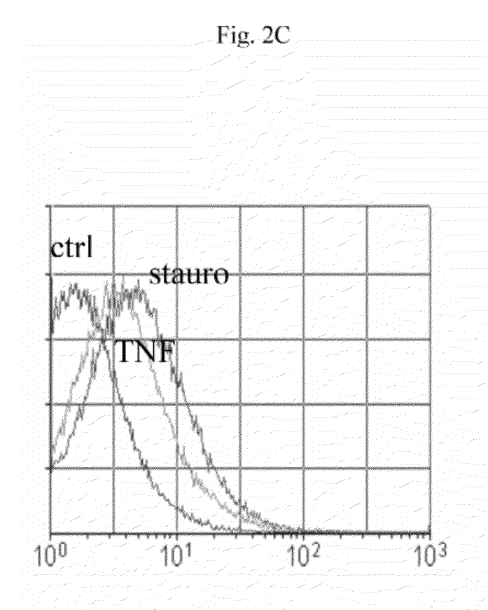

FIGS. 2A, 2B, and 2C show fluorescence profiles of the clone 10 population under various conditions (see examples);

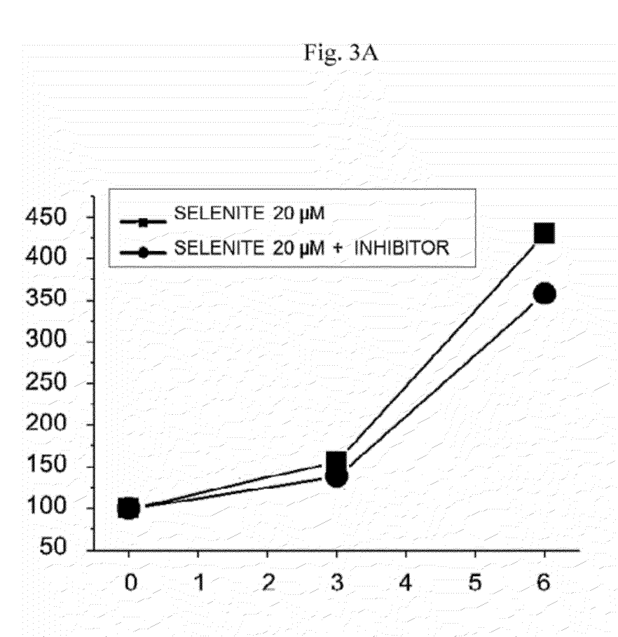

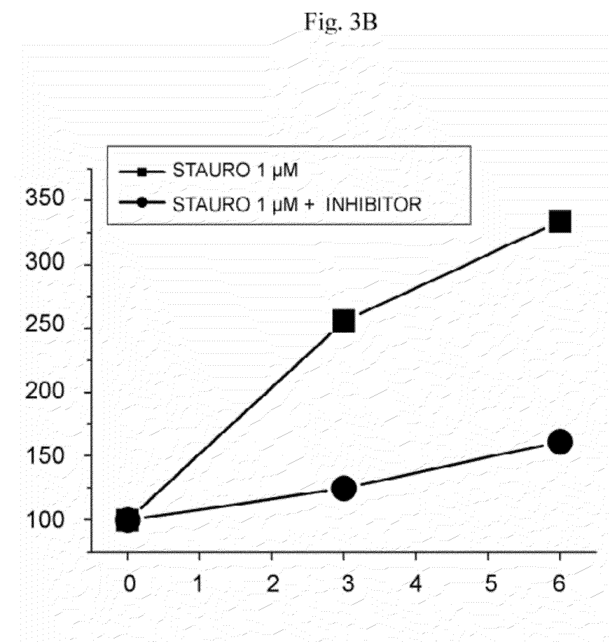

FIGS. 3A and 3B represent the variation of Bax activation under various conditions (see examples);

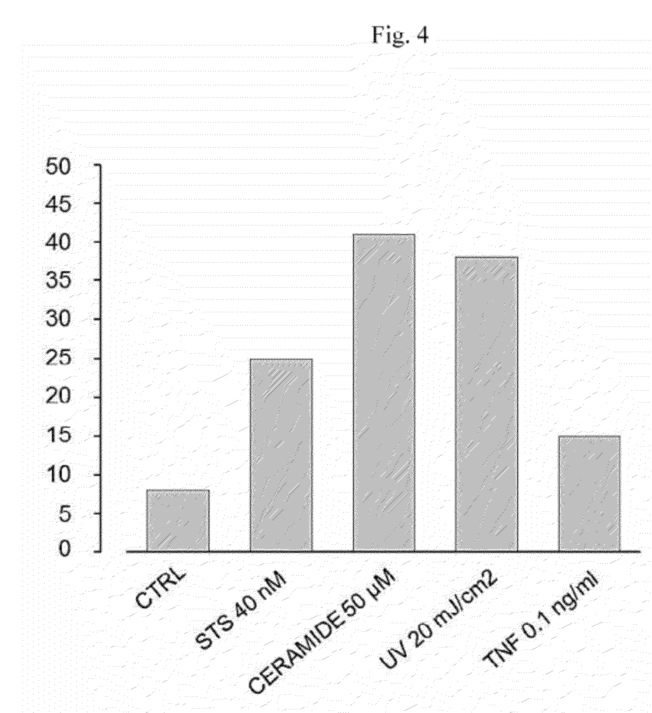

FIG. 4 represents a histogram of Bax activation under various conditions (see examples);

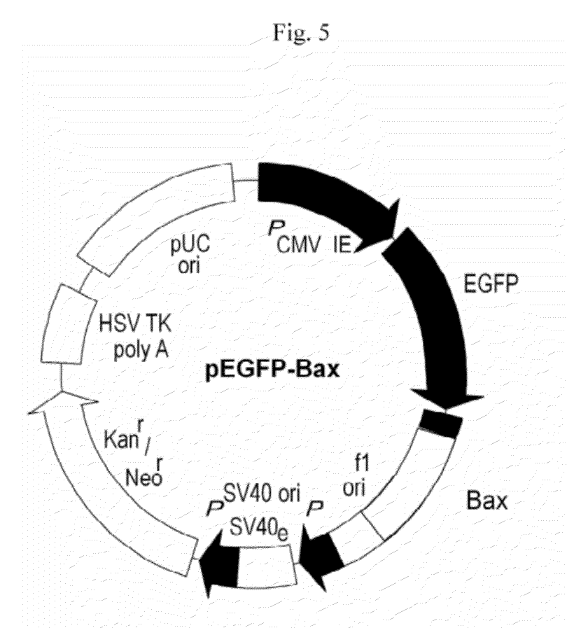

FIG. 5 represents the plasmid pEGFP-Bax;

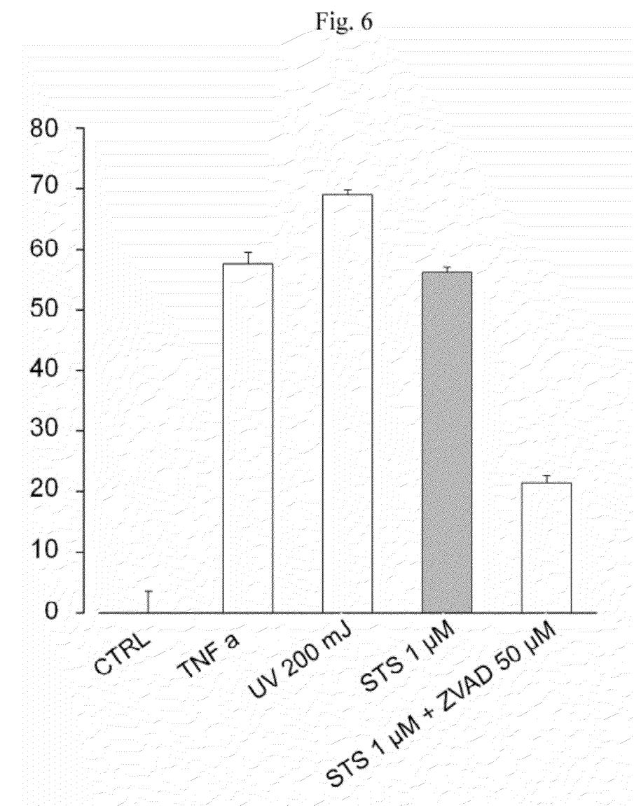

FIG. 6 represents the quantification of caspase 3;

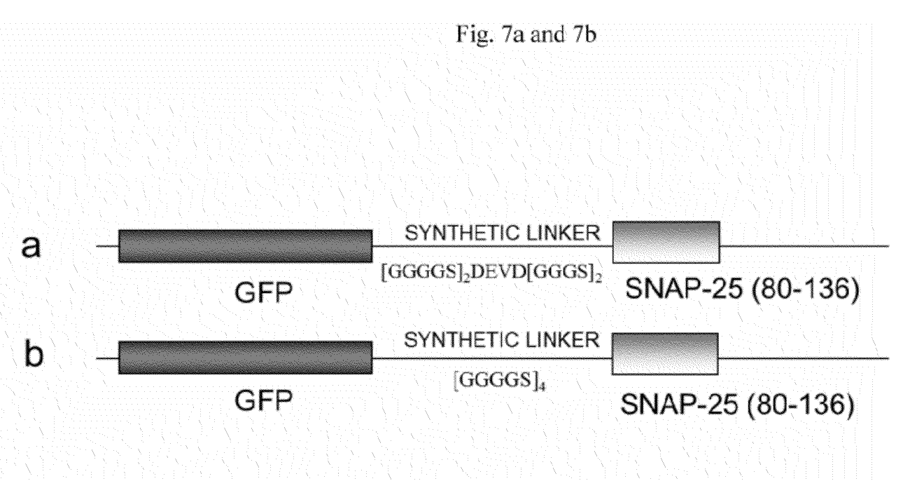

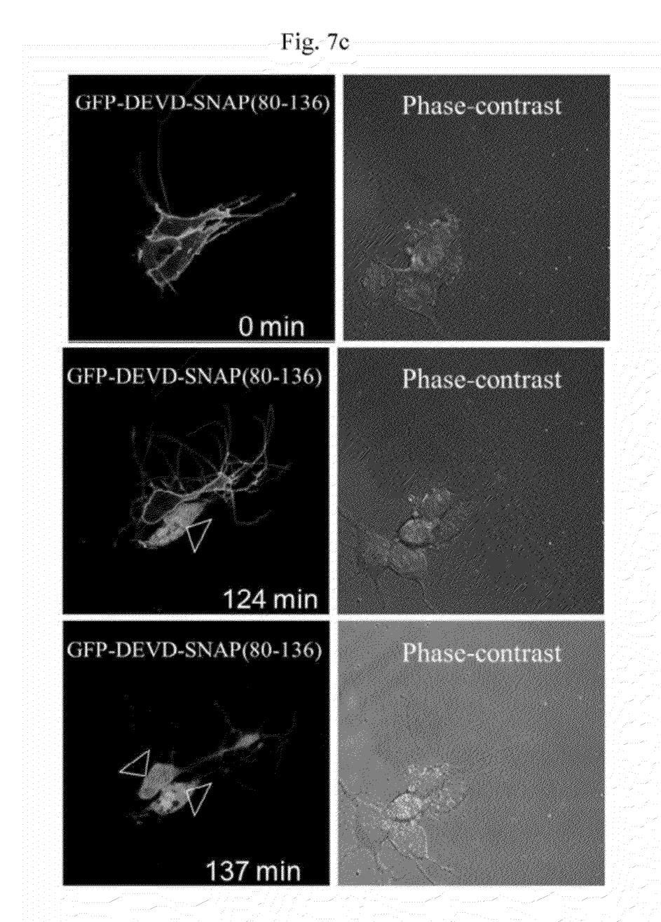

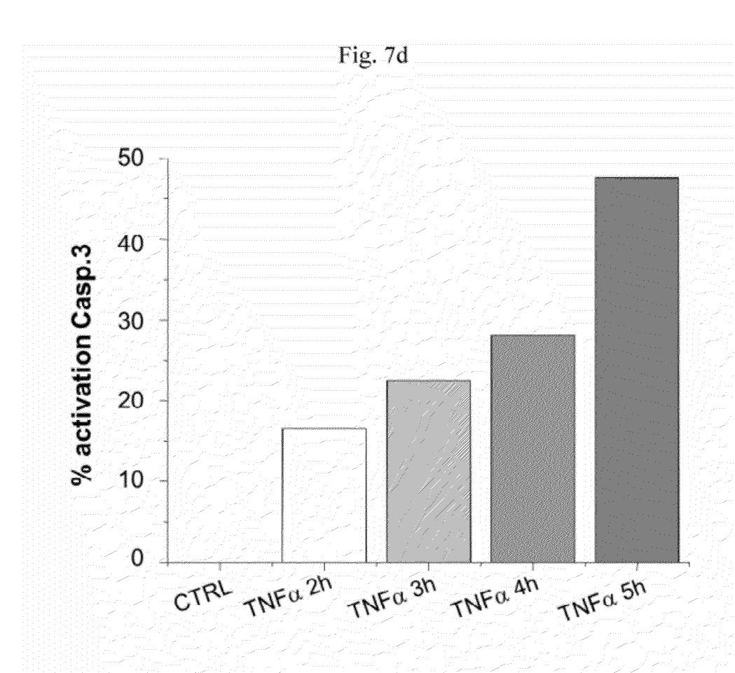

FIG. 7 illustrates the development of a caspase 3 probe anchored to the internal surface of the plasma membrane.

a) Schematic representation of the fusion protein GFP-DEVD-SNAP(80-136) and b) of non-cleavable control (same fusion protein without a consensus site for the protease). c) Release of the fluorescent protein in the cytosol following its cleavage observed by confocal microscopy in two cells of human SH-SY5Y neuroblastoma transfected transiently and treated with staurosporine. d) Measure by flow cytometry of the percentage of cells presenting an activated caspase 3 in a population of human HeLa cells transfected transiently with the protein GFP-DEVD-SNAP(80-136).

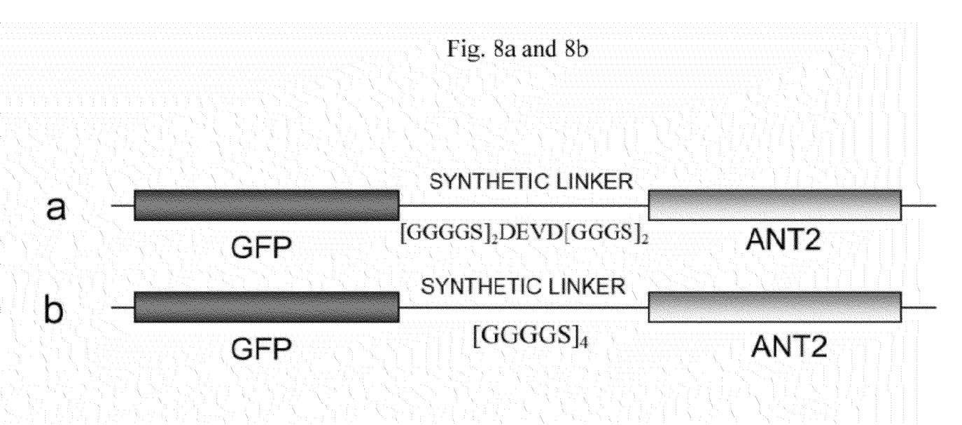

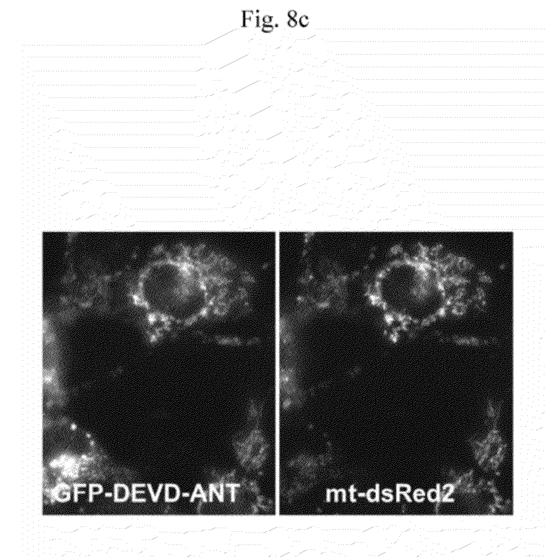

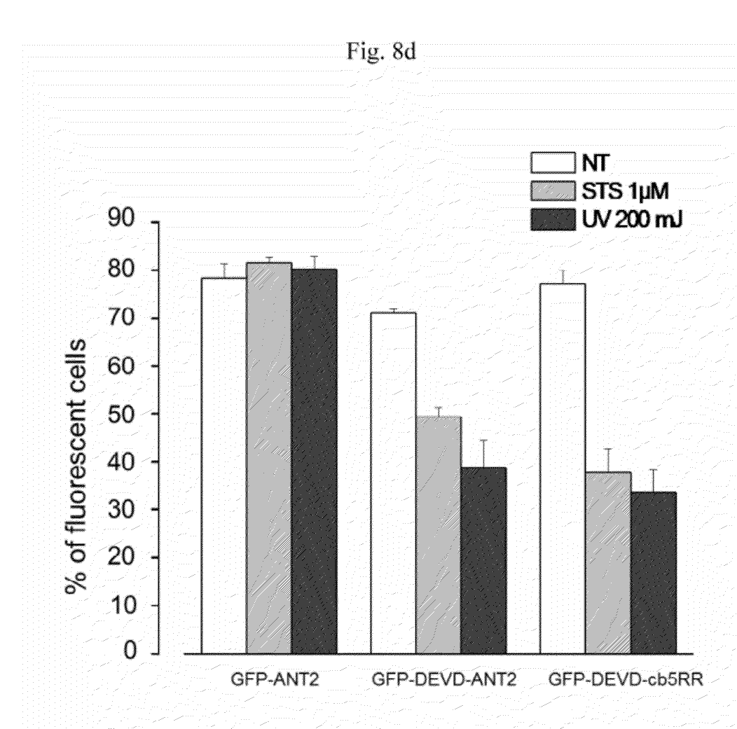

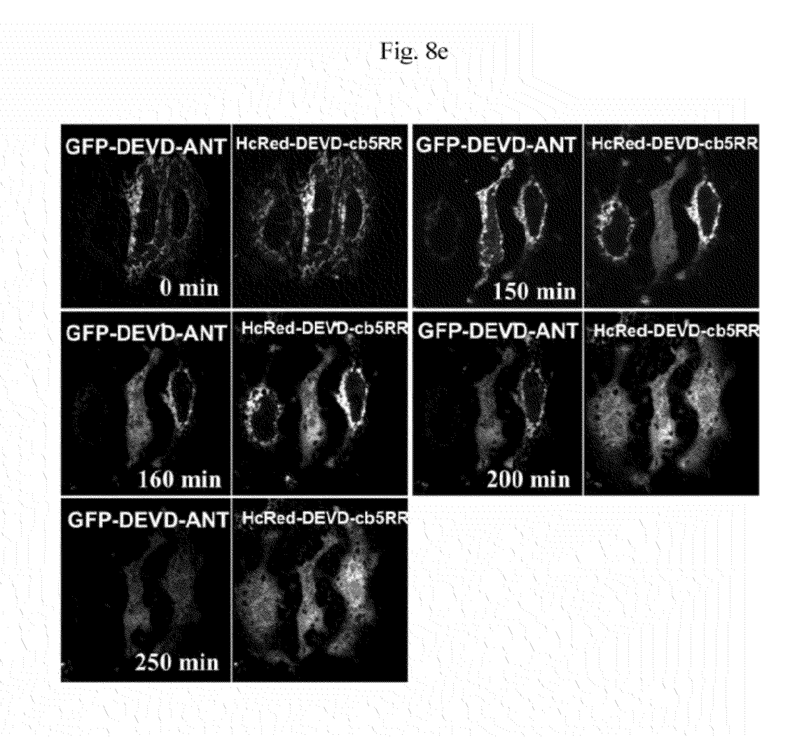

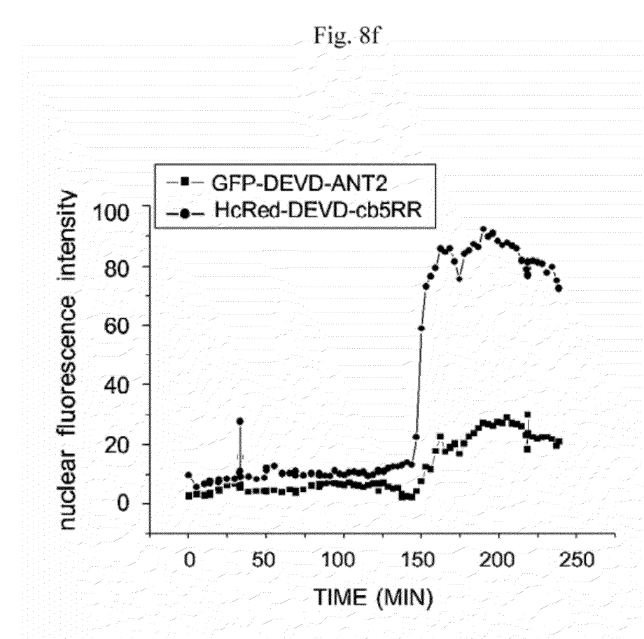

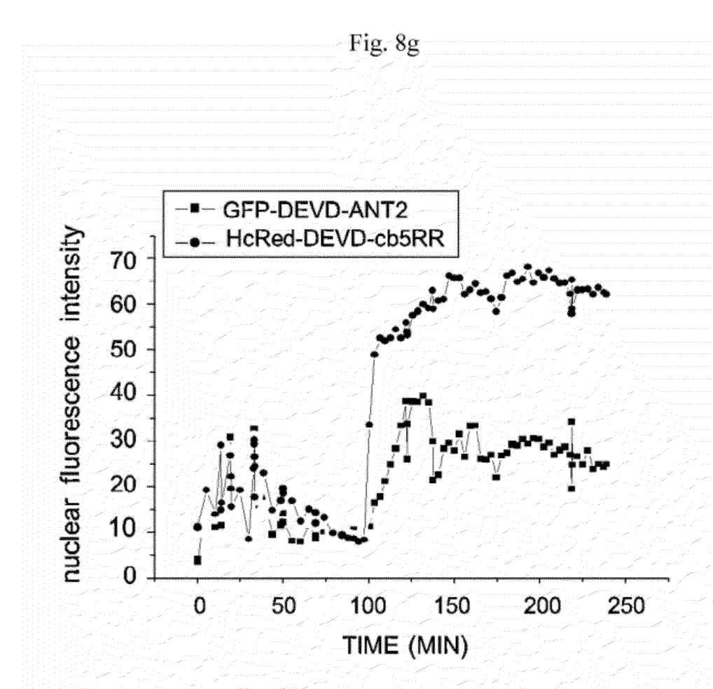

FIG. 8 illustrates the development of a caspase 3 probe anchored to the external surface of the internal mitochondrial membrane.

a) Schematic representation of the fusion protein GFP-DEVD-ANT2 and b) of non-cleavable control (same fusion protein without a consensus site for the protease).

c) Mitochondrial localization of the fusion protein (GFP-DEVD-ANT) in simian COS-7 cells co-transfected with a specific mitochondrial marker (mt-dsRed2). d) Detection by flow cytometry of the cleavage of the fusion protein GFP-DEVD-ANT2 compared to the non-cleavable control and to the GFP-DEVD-cb5TMRR protein in the HeLa cells transiently transfected in which apoptosis has been induced by various stimuli (1 μM staurosporine or 200 mJ/cm² UV). e)-f) Release of the fluorescent protein in the cytosol following its cleavage observed by confocal microscopy in two human HeLa cells transiently co-transfected with GFP-DEVD-ANT and HcRed-DEVD-Cb5RR and treated with staurosporine. The quantification of cleavage and of the diffusion of the fluorescent signal in the cytosol is carried out by measuring the increase in the respectively green and red fluorescent signal in a region of the nucleus.

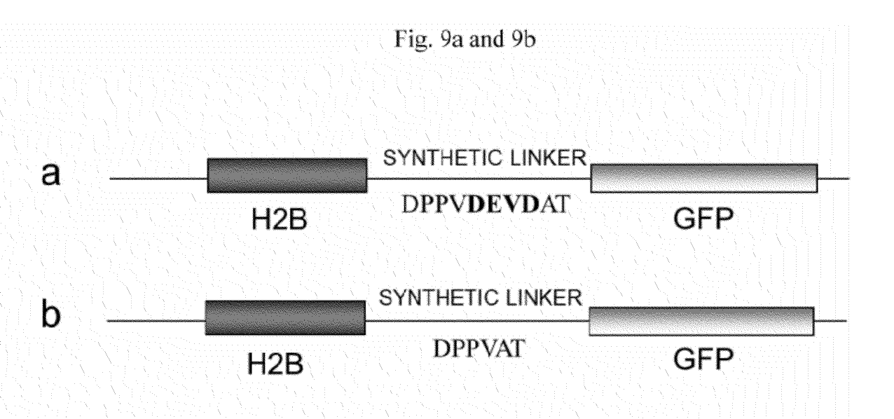

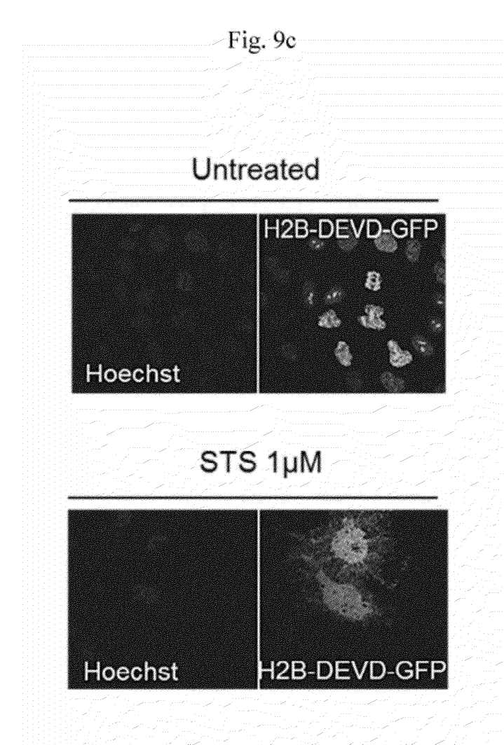

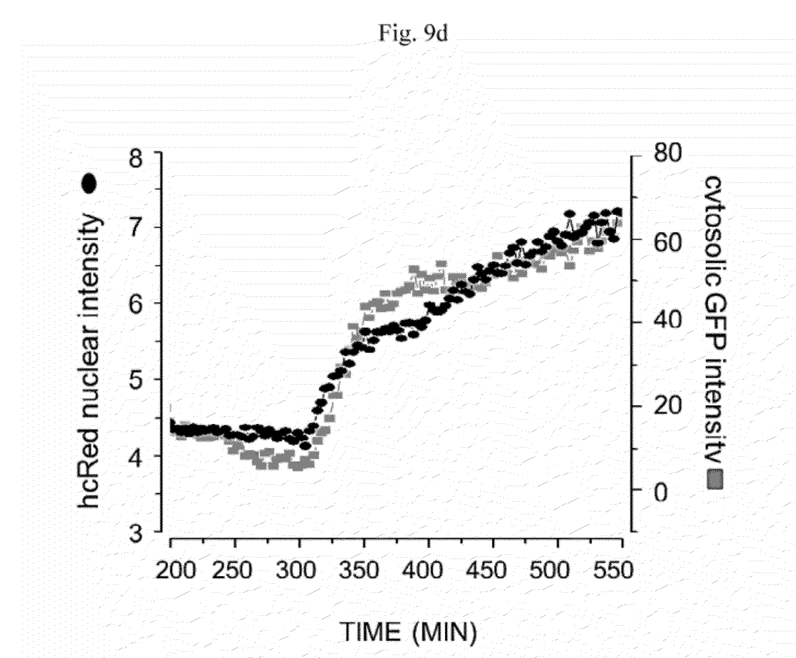

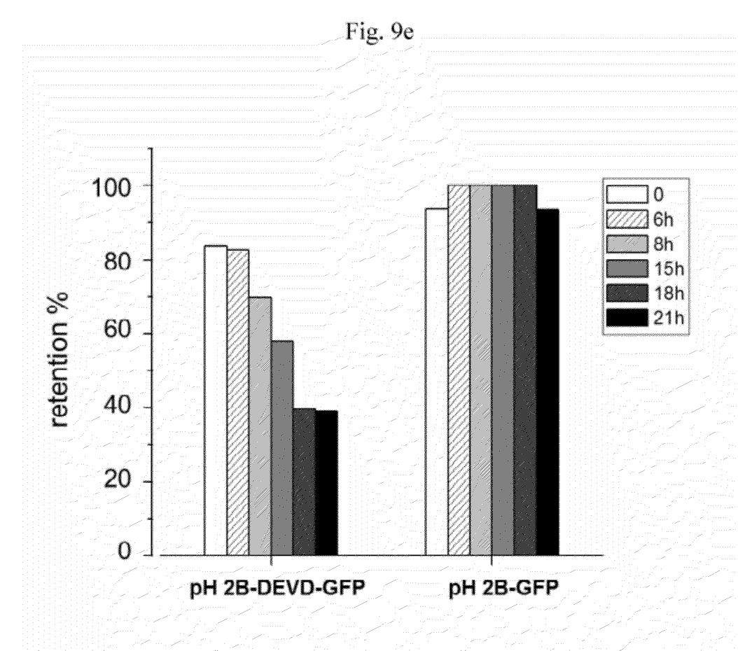

FIG. 9 illustrates the development of a caspase 3 probe with nuclear anchoring.

a) Schematic representation of the fusion protein H2B-DEVD-GFP and b) of non-cleavable control (same fusion protein without a consensus site for the protease). c) Distribution of the fluorescent protein in human HeLa cells transiently transfected: on the left, untreated cells (nuclear distribution), on the right, cells treated with staurosporine (cytosolic distribution). d) Release of the fluorescent protein in the cytosol following its cleavage, observed by confocal microscopy of a HeLa cell transiently co-transfected with H2B-DEVD-GFP and HcRed-DEVD-Cb5RR (caspase 3 probe anchored to the external mitochondrial membrane) and treated with staurosporine. The quantification of the cleavage and of the diffusion of the fluorescent signal respectively from the nucleus to the cytosol for the protein H2B-DEVD-GFP and from the cytosol to the nucleus for the protein HcRed-DEVT-cb5 is carried out by measuring the increase in the fluorescent signal, respectively green in a cytosolic region of the cell and red in a region of the nucleus. e) Evaluation by flow cytometry of the functionality of the probe by quantification of the percentage of retention of fluorescence after permeabilization in a population of HeLa cells transfected transiently with the protein H2B-DEVD-GFP and its non-cleavable control.

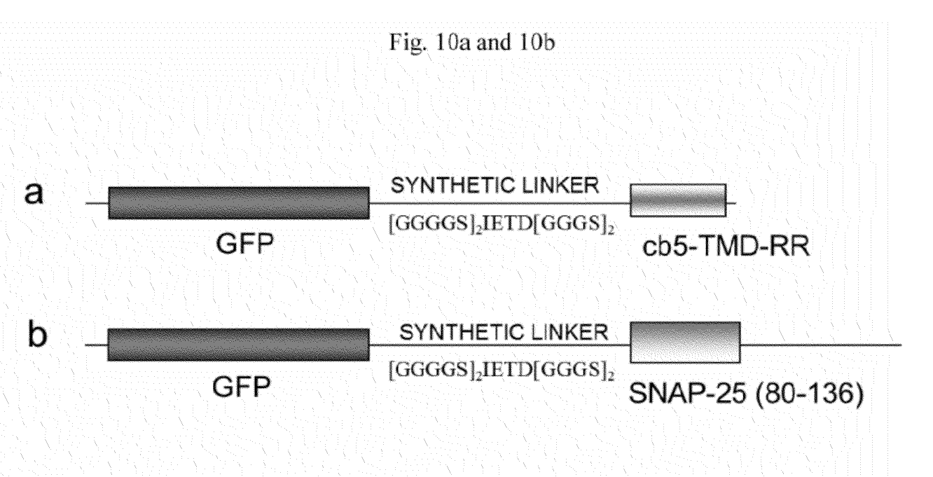

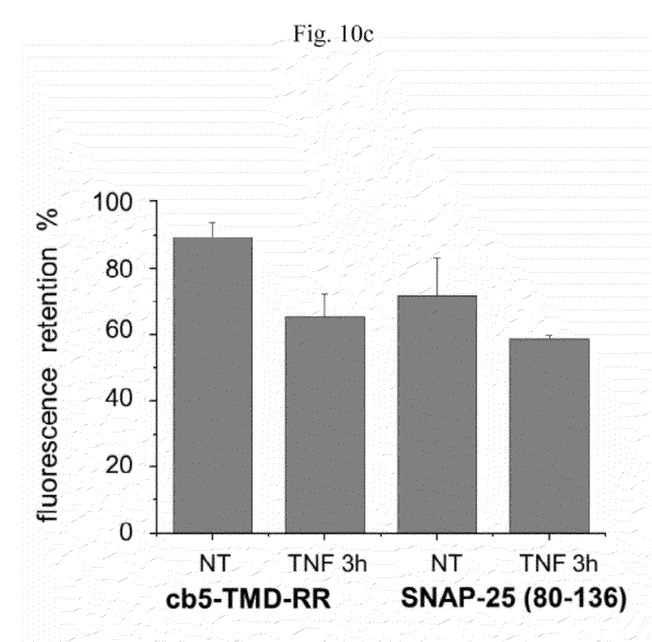

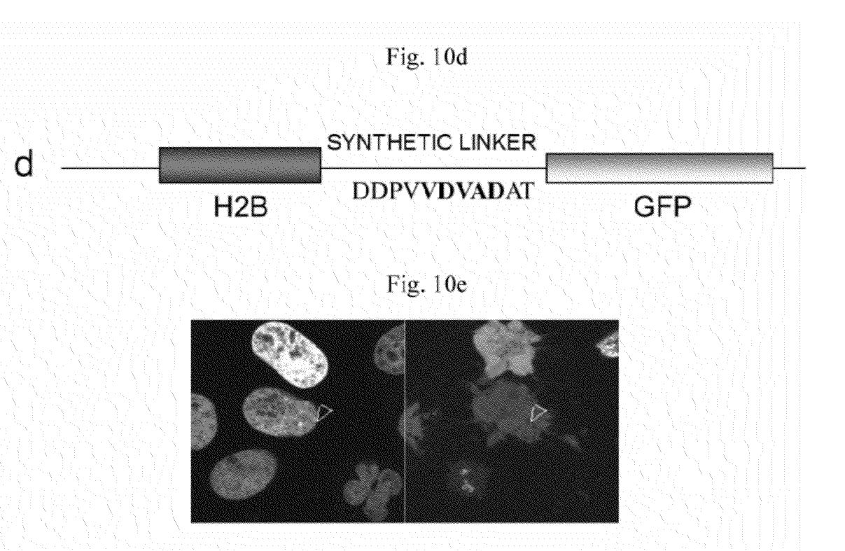

FIG. 10 illustrates the development of probes for measuring the activity of caspases 8 and 2.

a) Schematic representation of the fusion protein GFP-IETD-cb5-TMD-RR and b) GFP-IETD-SNAP(80-136). c) Evaluation by flow cytometry of the functionality of the probe by quantification of the percentage of retention of fluorescence in a population of HeLa cells transfected transiently with the GFP-IETD-cb5-TMD-RR protein and GFP-IETD-SNAP(80-136) and treated with TNF-α. d) Schematic representation of the fusion protein GFP-IETD-H2B. e) Distribution of the fluorescent protein in HeLa cells transfected transiently: on the left, untreated cells (nuclear distribution), on the right, the same cells treated with staurosporine for 3 hours (cytosolic distribution).

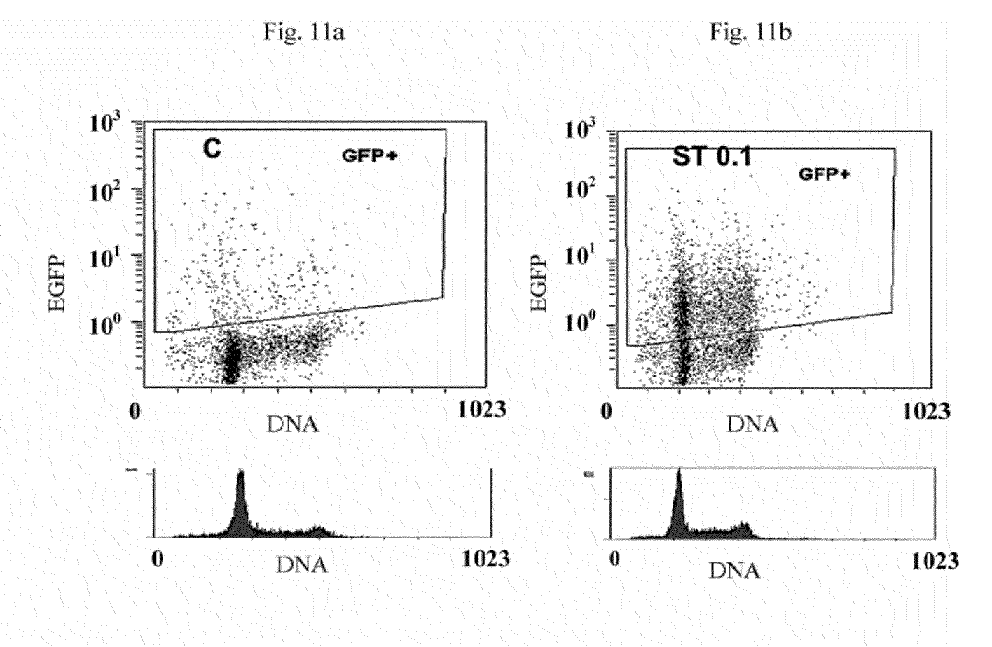

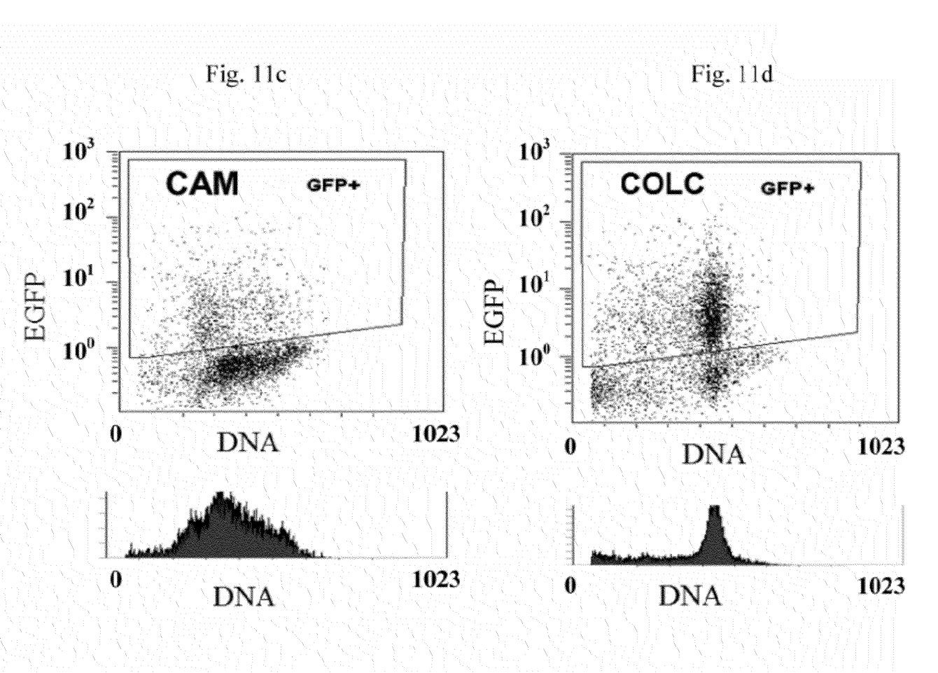

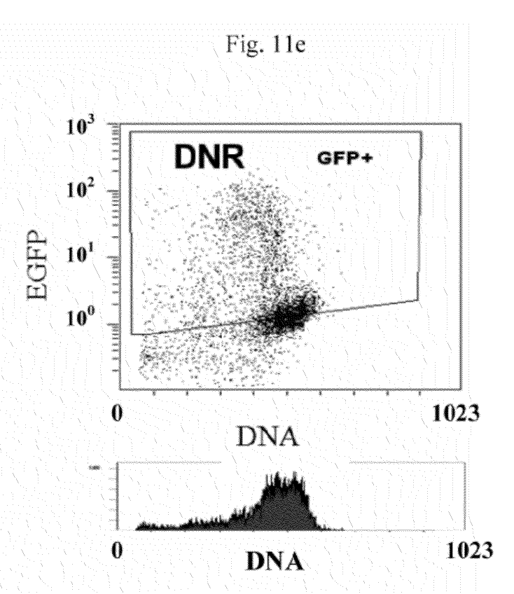

FIG. 11A-E represents the coupled measurement of Bax activation and the cell cycle. The clone 10 cells stably expressing the GFP-Bax fusion protein are treated with various drugs acting as pro-apoptotic agents and/or cytostatic agents. The measurement of Bax activation is carried out as described previously by evaluation of the retention of fluorescence after permeabilization with digitonin. In the permeabilization buffer, propidium iodide (0.4-0.8 mg/ml) was added and the cells were incubated for 30 minutes at 4° C. The distribution of the cells in the various phases of the cell cycle is read on the basis of their intensity of fluorescence in the red (PI). The figure shows how the simultaneous reading in channels FL1 (GFP) and FL3 (DNA) makes it possible to simultaneously evaluate the pro-apoptotic effect of Bax activation and the effect on the cell cycle (modification of distribution in phases Gl, S, and G2/M). Moreover, it makes it possible to evaluate if Bax activation takes place in a preferential phase of the cell cycle. At the top, untreated control cells (C) and cells treated with staurosporine (ST 0.1) (no effect on the cycle or the induction of Bax activation in any phase of the cycle).

At the bottom, treatment with camptothecin (CAM) (blockage in phase S, Bax activation preferentially in phase Gl); treatment with colcemid (COLC) (accumulation in phase G2, Bax activation in all phases of the cycle); treatment with daunorubicin (DNR) (accumulation in phase G2, Bax activation in phase S).

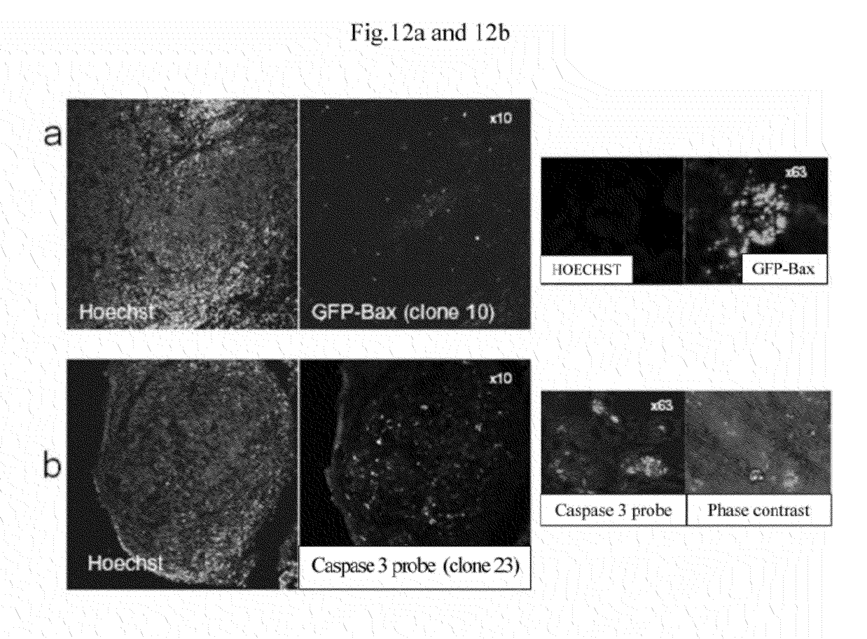

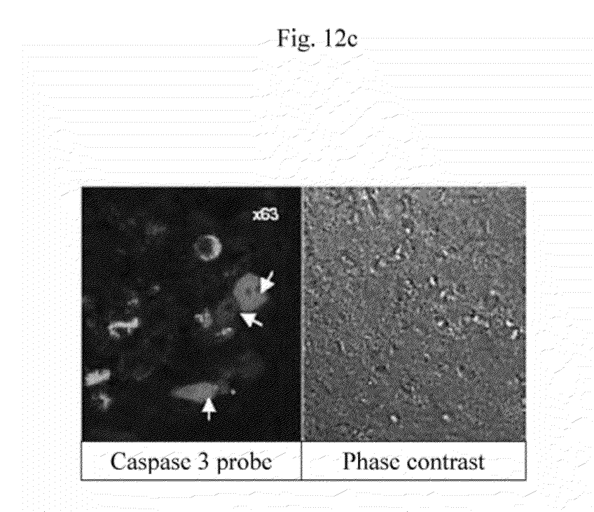

FIG. 12 represents the xenograft of lines expressing a fluorescent biosensor. a) Confocal microscopy image (10×) of a section of a solid tumor generated by a subcutaneous xenograft of clone 10 cells (stably expressing GFP-Bax) in nude mice. Marking of nuclei (Hoechst) and cells with activated Bax (GFP-Bax). On the right, the detail of a cell with GFP-Bax relocalized to the mitochondria (GFP-Bax) and corresponding nuclear marking (Hoechst). b) Confocal microscopy image (10×) of a section of a solid tumor generated by a subcutaneous xenograft of clone 23 cells (stably expressing the protein copGFP-DEVD-cb5TMD-RR) in nude mice. Marking of nuclei (Hoechst) and distribution of the biosensor (caspase 3 probe). On the right, the detail of the widest mitochondrial distribution of the recombinant probe. c) In a xenografted clone 23 tumor from a mouse treated with etoposide (40 mg/kg/day for 4 days), the appearance of cells having activated caspase 3 (arrows) (cytosolic distribution of fluorescence).

### EXAMPLE 1

### Activation of the Bax Protein

The following example describes a simple test that enables the detection of the conformational changes of the Bax protein during the induction of apoptosis.

As indicated previously, in a normal cell Bax is folded in way such that its very hydrophobic C-terminal end is protected by the rest of the molecule (2). During the induction of apoptosis, the protein undergoes a conformational change which modifies its properties, and the exposure of its C-terminal end induces a mitochondrial relocalization of Bax. In this form Bax behaves like a membrane protein inserted stably in the external mitochondrial membrane.

The use of a chimeric protein obtained by the fusion of GFP at the N-terminal end of Bax provides a recombinant fluorescent probe that indicates the localization of Bax.

The chimeric protein maintains the same properties as the native protein, in particular the capacity to undergo conformational change and to relocalize to the mitochondrion during the induction of apoptosis.

The model implemented uses a clone designated “clone 10”.

These are HeLa (human cervical tumor) cells which have been transfected using the calcium phosphate technique with a pEGFP-Bax plasmid coding for the chimeric fusion protein GFP-Bax under the control of the viral CMV promoter and which confers genetecin resistance.

The basic vector used is a commercial PEGFP-C3 vector (FIG. 5) from Clontech in which is inserted, under the control of the CMV promoter, the Bax cDNA fused in phase at its end 5′ with GFP cDNA lacking its stop codon.

Four days after transfection, the cells are exposed to a 1 mg/ml concentration of genetecin G418 which is gradually reduced to 0.1 mg/ml during the following week. After 2 weeks, a certain number of clones resistant to genetecin are isolated by selection under the fluorescence microscope.

Clone 10 contains an elevated percentage of uniformly fluorescent cells at the cytosolic level and this marking is stable over time (approximately 10 runs). The cells are maintained in culture in DMEM supplemented with 10% FCS, and 0.1 mg/ml of genetecin.

The test is then based on the observation, under the fluorescence microscope or by flow cytometry, of the cells in which Bax is not activated and thus is distributed uniformly in the cytosol and of the cells in which Bax has been activated following an induction of apoptosis. In the latter case, the fluorescent signal is aggregated around the mitochondria and the Bax protein is thus considered as “bound”.

To demonstrate the binding or the solubilization of Bax, the control population and the population treated with the apoptotic agent are treated with trypsin in order to detach them from their culture dish. The cells are then resuspended in an intracellular saline solution in the presence of 50 μM of digitonin and then analyzed by flow cytometry and the fluorescence of the GFP is measured in channel FL1.

Cells thus treated are represented in FIG. 1 and show that a very strong drop in intensity of fluorescence is observed in cells in which Bax has not been activated; initially the image is uniformly fluorescent and then this fluorescence mostly disappears after 300 seconds.

On the contrary, when Bax has been activated, it is noted that fluorescence is redistributed to the mitochondria and resists permeabilization, as can be determined from quantitative measurements taken from the corresponding curves.

FIG. 2 shows in FL1 various profiles of fluorescence of the clone 10 population, with and without permeabilization, in the presence of various pro-apoptotic agents.

“A” shows the fluorescence profile of the clone 10 population controls, with and without permeabilization. As the displacement of the distribution peak towards the left indicates, the fluorescent signal is sensitive to treatment with digitonin.

“B” shows in FL1 the cytometric profile of a population treated with a pro-apoptotic agent (20 μM selenite, 6 h) that activates Bax. By comparing the profiles obtained, with or without the inducer, and after permeabilization, it can be observed that under the apoptotic inducer the fluorescent signal has become resistant to the permeabilization treatment.

In “C”, the same difference is observed under the induction of apoptosis by staurosporine (1 μM, 6 h) or TNF-α (10 ng/ml, 6 h+10 μM CHX).

### EXAMPLE 2

### Quantification of the Relocalization of Bax to the Mitochondrion Under Apoptotic Induction

The technique makes it possible to evaluate the induction of Bax by measuring the percentage of cells in which the relocalization of Bax to the mitochondrion has taken place.

With this approach, it is possible:
to analyze if the expression of a gene induces Bax activation or not and
to quantitatively evaluate the pro-apoptotic or cytoprotective capacity of a drug.

To evaluate an exogenous gene's capacity to activate Bax, clone 10 is transfected with the cDNA of the protein of interest in association with another cDNA coding for a fluorescent marker spectrally differentiable from GFP and having a subcellular, membrane, or compartmentalized (resistant to permeabilization) localization, for example DsRed designated for the mitochondrial matrix (mtDsRed). In the latter case, after permeabilization, the intensity of fluorescence recorded in channel FL1 (GFP) always indicates the level of Bax activation, while the intensity of the signal in FL3 (DsRed) indicates whether the cell overexpresses the protein of interest or not.

By performing a bi-parametric measurement on clone 10, it is thus possible to correlate Bax activation to the overexpression of a protein of interest.

The results indicated in FIGS. 3A and 3B shows the percentage of a cell population that underwent the relocalization of Bax to the mitochondria following a pharmacological treatment.

FIG. 3A represents the development over time of a cell population treated with 20 μM selenite in the absence (curve D) and in the presence (curve E) of an inhibiter and with 40 nM of staurosporine in the absence and in the presence of the same inhibiter B.

Thanks to this technique, it is observed that the kinetics of Bax activation are not modified by the inhibiter during the treatment with selenite, whereas on the contrary this inhibiter is active on the staurosporine.

Similarly, the histogram in FIG. 4 represents the percentage of the cells in which Bax has been activated after treatment for 24 h with 40 nM of staurosporine, 50 μM of ceramide, or 0.1 ng/ml of TNF in the presence of cycloheximide, under the influence of UV radiation.

This technique thus makes it possible to distinguish the pro-apoptotic properties of various agents.

It presents many advantages with respect to the currently available techniques for evaluating Bax activation and its relocalization to the mitochondria which are based on subcellular fractionations and Western blot quantification of the quantity of Bax proteins present in the various fractions or by immunofluorescence with antibodies which specifically recognize the protein that underwent the conformational change of activation.

Compared to these techniques, the approach described previously requires only one much reduced experimental manipulation (the cumulative time is less than 30 min compared to 24 h for the other techniques), it allows the measurement of the parameter of interest, even within the cell, without inducing artifacts related to fractionation or to fixation and the use of detergents in large quantities (immunofluorescence), and it presents low experimental cost.

### EXAMPLE 3

### Measurement of Caspase 3 Activity During Apoptosis

The principal effectors of apoptosis are caspases, which are cysteine proteases characterized by an absolute specificity for an aspartate in position P1 in their cleavage site. All of these enzymes contain an identical pentapeptide sequence in their active site and participate, with other proteases such as calpain, in the many proteolytic events which occur in a cell during apoptosis, leading to the cleavage of the protein substrates that play a key role in normal cellular functions (cytoskeleton proteins, nuclear proteins, or DNA repair enzymes).

Caspase activation can take two principal pathways. The first is the “mitochondrial” pathway in which protein Apaf-1 interacts with procaspase-9, in the presence of dATP and cytochrome c, released from the mitochondrial intermembrane space, to form the “apoptosome”, thus enabling the activation of caspase-9 (autocatalytic cleavage of procaspase-9) then of caspase-3. The other pathway is that of the receptors of the superfamily of TNF receptors on the plasma membrane. The interaction of TNFR1 or Fas (CD95 or APO-1) with their natural ligand or a monoclonal antibody agonist enables the assembly of a multi-protein cytoplasmic complex called DISC (death-inducing signaling complex) and the initiation of the apoptotic cascade by the activation of procaspase-8.

Currently a limited number of methods exist which have already been mentioned previously and none is perfectly satisfactory to measure caspase activation.

Within the framework of the present invention, a new recombinant probe is used which is appropriate for microscopy and cytometry for measuring caspase activity in apoptotic cells.

This new probe consists of a fusion protein in which a fluorescent protein, such as DsRed2 or EGFP, is connected by a short linker containing the caspase 3 consensus site to a transmembrane sequence ensuring the specific anchoring of the probe to the external mitochondrial membrane.

The corresponding sequence is described in SEQ ID No. 1.

The underlined portion corresponds to the synthetic linker containing the caspase 3 cleavage sequence, then the transmembrane domain of the mutated cytochrome b5 to which has been conferred specific mitochondrial addressing.

During the induction of apoptosis, the activated caspase 3 cleaves the DEVD sequence contained in the linker that had been interposed between the fluorescent protein and the transmembrane sequence. The previously-bound GFP becomes a soluble protein in the cytosol of the cell.

The signal bound in the cells in which caspase 3 is not activated becomes soluble in the cells in which caspase is activated.

The cells are cultivated on glass slides and transfected with the vector described previously; they are then mounted in a saline medium in an incubation chamber and observed under the fluorescence microscope. Before or during the observation, they are treated with the pro-apoptotic agent. The activation of caspase 3 and its kinetics can be demonstrated by simple observation of the modification of the intracellular distribution of the fluorescent signal which quickly changes from mitochondrial to cytosolic once the caspase is activated.

But it is more convenient to measure caspase activation by flow cytometry.

The control population and the population treated with the apoptotic agent are detached from their culture dishes by trypsin treatment; the cells are then resuspended in an intracellular saline solution in the presence of 50 μM of digitonin. Next, the cells are analyzed by flow cytometry and the fluorescence of the GFP is measured in channel FL1.

The technique presented makes it possible to evaluate the activation of caspase 3 by measuring the percentage of cells in which the cleavage of the fluorescent sensor has taken place.

With this approach, it is thus possible:
to analyze if the expression of a gene induces the activation of caspase 3 or not and
to evaluate quantitatively the pro-apoptotic or cytoprotective capacity of a drug.

Thus, FIG. 6 represents the quantification of the action of caspase 3 in a population of HeLa cells treated with various apoptotic inducers: UV irradiation (200 mJ/cm²), 100 μg/ml of TNF-α, 1 μM of staurosporine for 6 h, and 1 μM of staurosporine in the presence of caspase inhibiter (ZVAD 50 μM).

The quantification of another apoptotic parameter is represented in parallel (mitochondrial depolarization which in this apoptosis induction model depends on caspase activation).

Of course, this method is directly generalizable to all caspases and other proteases which can be introduced into various systems and for which those skilled in the art will be able to create the corresponding vector.

### EXAMPLE 4

### Other Subcellular Probe Anchoring Strategies

The caspase 3 probe described above is based on membrane anchoring consisting of a short transmembrane domain which is embedded in the external mitochondrial membrane (mutant cytochrome b5 C-terminal segment). This protein domain thus confers to the fusion protein a mitochondrial distribution and the property of being resistant with respect to a selective permeabilization of the plasma membrane. It is shown in this example that the principle of the test is extendible to other subcellular anchoring strategies which imply a resistance of the fluorescent signal to the permeabilization of the plasma membrane. The type of anchoring can not necessarily be represented by a transmembrane domain itself but quite simply by a protein domain which, by its molecular interactions or its post-translational modifications, confers on the fluorescent protein to which it is fused an “anchored” but potentially diffusible state in the cytosol or in extracellular environment after cleavage by the protease of interest. Moreover, the specific subcellular localization (plasma membrane, nucleus, intermembrane mitochondrial space) can give additional information on the accessibility of substrates to proteases and, therefore, on the intracellular localization of proteolytic activities.

### EXAMPLE 4a

### Caspase 3 Probe Anchored to the Internal Surface of the Plasma Membrane

In this fusion protein, the intracellular anchoring domain is comprised of a portion of the murine SNAP-25 protein. SNAP-25 is a protein implied in secretary vesicle fusion processes and it is located on the cytosolic surface of the plasma membrane by palmitoylation of three cysteine residues.

The minimal SNAP-25 palmitoylation domain, which is constituted by amino acids 80-136 (SEQ ID No. 4), has been isolated and it has been fused to the fluorescent protein via the linker containing the caspase 3 cleavage site.

This fusion protein is thus located in the plasma membrane and its fluorescent signal is resistant to permeabilization by digitonin. The proteolytic activity of caspase 3 cleaves the linker, causes a redistribution of fluorescence in the cytosol, and the signal is lost after permeabilization (FIG. 7). In flow cytometry, this probe gives results completely comparable with the probe anchored to the external mitochondrial membrane previously described.

### EXAMPLE 4b

### Caspase 3 Probe Anchored to the External Surface of the Internal Mitochondrial Membrane

In this second example, the protein domain used to anchor the probe is represented by the entire sequence of the mitochondrial adenine translocator (ANT2) (SEQ ID No. 8). This is an integral protein of the internal mitochondrial membrane whose N-terminal end is exposed in the intermembrane space. The fluorescent protein with its cleavable linker has thus been fused at this end.

It has thus been shown that: (i) this fusion protein is properly located in the mitochondrion; (ii) the fluorescent protein is cleavable by caspase 3; and (iii) the fluorescence signal becomes sensitive to permeabilization after the cleavage of caspase 3 (FIG. 8).

This fusion protein thus behaves well as a probe for the measurement of caspase 3 activity, according to the measurement principle described in the present application. This example also shows that the method according to the invention makes it possible to obtain additional information on the spatial distribution of the proteolytic activity studied: in this case, although trapped in the intermembrane space, the probe can be cleaved by caspase 3, which shows that this intracellular space becomes accessible during apoptosis to cytosolic proteins such as caspase 3.

### EXAMPLE 4c

### Caspase 3 Probe with Nuclear Anchoring

This third example shows that intracellular anchoring can be obtained by the effect of highly stable protein-protein and protein-nucleic acid interactions.

A caspase 3 probe with nuclear localization was obtained by fusing the fluorescent protein with the histone protein 2b via the cleavable linker by caspase 3. The control fusion protein H2B-GFP (without a specific sequence for caspase 3) is located very stably in the nucleus: by way of its interaction with chromatin (DNA) within the nucleosomes (multi-protein complexes), it does not diffuse, as the resistance of the fluorescent signal during permeabilization of the plasma membrane demonstrates. During apoptosis, even in the late stages that involve the degradation of DNA caused by internucleosomal cleavage of chromatin by endonucleases specific to apoptosis, the fluorescent protein remains trapped in the nucleosomes and its distribution follows the distribution of chromatin by the indication of the pycnotic nuclei characteristic of apoptosis.

The protein H2B-DEVD-GFP, in which the caspase 3 cleavage sequence was inserted in the linker between the histone (SEQ ID No. 10) and the GFP, behaves in the same manner as the control probes in the untreated cells. On the other hand, in the cells treated with an apoptotic inducer, the fluorescence of the GFP diffuses outside the nucleus during the activation of caspase 3 and becomes distributed uniformly in the cytoplasm. This fluorescence becomes sensitive to the permeabilization of the plasma membrane. The permeabilization experiments show that this protein behaves well as a probe that enables the measurement of the activity of caspase 3 in the cell nucleus (FIG. 9).

### EXAMPLE 5

### Application of the Principle of the Method According to the Invention to Probes Measuring Other Proteolytic Activities

The measurement approach developed for caspase 3 was extended to two other proteases implied in programmed cell death.

Fusion proteins GFP-cb5TMRR and GFP-SNAP(80-136) were constructed which carry, in the linker connecting the fluorescent protein and the anchoring segment, the caspase 8 consensus sensing site (IETD) which is principal “initiating” pro-apoptotic caspase. In the same manner, a fusion protein GFP-H2B was constructed which contains, in the sequence linker, the caspase 2 consensus sensing sequence (VDVAD), which is a protease with nuclear localization. The sensing components thus used have as sequences SEQ ID No. 6 and 14 (for caspase 8), and SEQ ID No. 12 (for caspase 2). These proteins are cleavable and the quantification of the proteolytic activity can be carried out via cytometry as shown for caspase 3 (FIG. 10).

### EXAMPLE 6

### Examples of Application of the Test Using Flow Cytometry in Experimental In Vivo Tumor Models

In this example, two other applications of the probes and the method according to the invention are demonstrated: the first relates to the development of a coupled biparametric flow cytometry test, and the second describes the application of biosensors in the evaluation of anti-cancer activity in animal models.

### EXAMPLE 6a

### Measurement Coupled to that of the Cell Cycle

It is possible to apply the technique in double tests in which the measurement of the molecular event revealed by the specific probe is coupled with the measurement of another cellular parameter such as the cell cycle. The measurement of the cell cycle can be carried out in a traditional way by adding to the permeabilized cell suspension a sufficient concentration of propidium iodide (0.4-0.8 mg/ml). The cells are incubated for 30 minutes to allow time for PI to become intercalated in the genomic DNA, and then analyzed using flow cytometry by simultaneously measuring “green” fluorescence (FL1=the recombinant biosensor signal based on GFP or another fluorescent protein emitting in the green range) and “red” fluorescence (FL3=the intensity of PI corresponding to the chromatin content of each cell, which enables the cell cycle to be read).

The technique thus makes it possible to follow in a simultaneous way the distribution of the cell population in the various phases of the cycle and to follow the molecular event specifically detected by the probe. In particular, this technique offers two principal advantages:
it makes it possible to detect a possible relation between the activation of the molecular process studied and a given phase of the cell cycle;
used in the screening of candidate anti-cancer molecules, it makes it possible to identify simultaneously the compounds having a cytostatic activity only, the compounds having a pro-apoptotic activity only, and the compounds that are both cytostatic and pro-apoptotic.

FIG. 11 shows the effect of several pro-apoptotic drugs used as anti-cancer agents in the coupled test “activation of Bax/cell cycle” (see legend). In the same manner, the coupled test “activation of caspase 3/cell cycle” (not shown) was carried out successfully.

### EXAMPLE 6b

### Application of Biosensors in the Evaluation of Anti-Cancer Activity In Vivo in Mice Carrying a Xenograft Tumor

The subcutaneous xenograft of human tumor cell lines in “nude” mice (deprived of cellular immunity) in vivo represents a classical preclinical model for the evaluation of the effectiveness of new molecules with anti-cancer potential. However, this evaluation is generally based on the simple measurement of the size and growth of established tumors, and thus does not allow connection of the “macroscopic” effect of the molecule tested with a precise molecular purpose.

The present example shows that the xenograft approach is applicable to cell lines which stably express the recombinant biosensors for measurement of the activation of Bax and of caspase 3.

These lines (designated clone 10 and clone 23, respectively), injected subcutaneously in “nude” mice form solid fluorescent tumors after a few days.

These in vivo tumors represent a new kind of model for the evaluation of the effectiveness of new molecules with anti-cancer potential since they give information about the specific molecular activity of the product tested at the level of tumor tissue (FIG. 12), all while permitting classical macroscopic morphometric measurements, which thus make it possible to correlate a “molecular effect/effect on the tumor growth” in vivo.

### BIBLIOGRAPHY

1) Ferri K. F., Kroemer G. (2001). Organelle-specific initiation of cell death pathways. Nat. Cell. Biol. 3(11):E255-63
2) Suzuki M., Youle R. J., Tjandra N. (2000). Structure of Bax: coregulation of dimer formation and intracellular localization. Cell 10; 103(4):645-54

1. A method for the demonstration of the occurrence of the activation of a protease in a cell containing a marker protein, said marker protein being a fusion protein containing a fluorescent fragment and being used as a direct or indirect marker for the occurrence of the activation of a protease, wherein:
a) said marker protein is initially bound by way of subcellular anchoring or by compartimentalization at the subcellular level;
b) the cell is subjected to a permeabilization of the plasma membrane; and
c) the presence of a solubilized marker protein, that is in the form of a free protein, which was initially bound is detected in the extracellular medium;
said detection being made by any appropriate means, making it possible to determine if solubilization, and thus the corresponding activation of a protease, has occurred.

2. A method according to claim 1, wherein the marker protein is produced in the cell by an expression vector.

3. A method according to claim 1, wherein the marker protein is constitutively produced by the cell.

4. A method according to claim 1, wherein said detection is made by flow cytometry or fluorescence microscopy on the cells after permeabilization of the plasma membrane.

5. A method according to claim 1, wherein the activation of a protease leads to the cleavage or the modification of the marker protein and solubilizes it.

6. A method according to claim 1, wherein to determine if the activation of a protease has occurred, the marker protein is a fusion protein containing the protease cleavage site and, on either side, a subcellular anchoring site, preferably a membrane anchoring site, and a fluorescent protein, and wherein, when the protease is expressed, the marker protein is solubilized by cleavage and the fluorescent protein is released.

7. A method according to claim 1, wherein the protease is a caspase.

8. A method according to claim 1, wherein said marker contains a sensing component which will undergo solubilization.

9. A method according to claim 8, wherein the sequence of said sensing component is coded by a nucleic acid comprising a sequence chosen among sequences SEQ ID NOS: 1, 3, 5, 7, 9, 11, and 13.

10. A method according to claim 8, wherein the sequence of said sensing component includes a sequence chosen among SEQ ID NOS: 2, 4, 6, 8, 10, 12, and 14.

11. A method according to claim 1, wherein the demonstration of the activation of a protease is coupled with the measurement of the cell cycle.


SEQUENCE LISTING (14 sequences)

SEQ ID NO: 1 - DNA, 195 bp, Artificial sequence
  Probe
gaaggtggaggaggttcagatgaagtcgattcaggaggaggtggatctggaggtggcggatccttcgagccgtccgaaactctgatcactaccgttgaatcgaactcgagttggtggactaactgggttatccctgcgatctctgctctggttgtagcgctgatgtaccggcgttaatgactgcagtctagaggg

SEQ ID NO: 2 - protein, 58 aa, Artificial sequence
  Probe
EGGGGSDEVDSGGGGSGGGGSFEPSETLITTVESNSSWWTNWVIPAISALVVALMYRR

SEQ ID NO: 3 - DNA, 294 bp, Artificial sequence
  Caspase 3 probe DEVD-SNAP-25(80-136)
gaaggtggaggaggttcagatgaagtcgattcaggaggaggtggatctggaggtggcggatccttcgagccgtccgaaactctgatcactaccgttgaatcgaactcgagtatggacctaggaaaattctgcgggctttgtgtgtgtccctgtaacaagcttaaatccagtgatgcttacaaaaaagcctggggcaataatcaggatggagtagtggccagccagcctgcccgtgtggtggatgaacgggagcagatggccatcagtggtggcttcatccgcagacgcgtctaa

SEQ ID NO: 4 - protein, 97 aa, Artificial sequence
  Caspase 3 probe DEVD-SNAP-25(80-136)
EGGGGSDEVDSGGGGSGGGGSFEPSETLITTVESNSSMDLGKFCGLCVCPCNKLKSSDAYKKAWGNNQDGVVASQPARVVDEREQMAISGGFIRRRV

SEQ ID NO: 5 - DNA, 294 bp, Artificial sequence
  Caspase 8 probe IETD SNAP-25(80-136)
gaaggtggaggaggttcaattgaaaccgattcaggaggaggtggatctggaggtggcggatccttcgagccgtccgaaactctgatcactaccgttgaatcgaactcgagtatggacctaggaaaattctgcgggctttgtgtgtgtccctgtaacaagcttaaatccagtgatgcttacaaaaaagcctggggcaataatcaggatggagtagtggccagccagcctgcccgtgtggtggatgaacgggagcagatggccatcagtggtggcttcatccgcagacgcgtctaa

SEQ ID NO: 6 - protein, 97 aa, Artificial sequence
  Caspase 8 probe IETD SNAP-25(80-136)
EGGGGSIETDSGGGGSGGGGSFEPSETLITTVESNSSMDLGKFCGLCVCPCNKLKSSDAYKKAWGNNQDGVVASQPARVVDEREQMAISGGFIRRRV

SEQ ID NO: 7 - DNA, 960 bp, Artificial sequence
  Caspase 3 probe DEVD-ANT-2
gaaggtggaggaggttcagatgaagtcgattcaggaggaggtggatctggaggtggcggatccatgacagatgccgctgtgtccttcgccaaggacttcttggccggtggagtggccgcagccatctccaagacagcggtagcacccatcgagagggtcaagctgctgctgcaggtgcagcatgccagcaagcaaatcacggcagataagcaatacaagggcatcatagactgcgtggttcgtatccccaaggaacagggagtcctgtccttctggcgtgggaacctggccaatgtcatcagatacttccccacccaggctctcaactttgccttcaaagataaatacaagcagatctttctgggtggtgtggacaagaggacccagttctggcgctactttgcagggaacctggcatcaggtggtgccgctggggctacatccttgtgctttgtgtaccctcttgattttgcccgtacccgtctagcagctgatgtgggcaaagctggagctgaaagggaattcaaaggccttggtgactgcctggttaagatctacaaatctgatgggattaagggcctgtaccaaggctttaatgtgtcagtacagggcattatcatctaccgagctgcctactttggtatctatgacactgcaaagggaatgctcccagatcccaagaatactcacatcttcatcagctggatgattgcacagtctgtcactgctgtcgctggcctgacttcctatccttttgacacggttcgccgtcgtatgatgatgcagtctggacgcaaaggaactgatatcatgtacacaggcacgcttgactgctggcggaagatcgcgcgcgatgaagggagcaaggcttttttcaagggcgcatggtccaacgttctcagaggcatgggtggcgcctttgtgcttgtcttgtatgatgagatcaagaaatacacataa

SEQ ID NO: 8 - protein, 319 aa, Artificial sequence
  Caspase 3 probe DEVD-ANT-2
EGGGGSDEVDSGGGGSGGGGSMTDAAVSFAKDFLAGGVAAAISKTAVAPIERVKLLLQVQHASKQITADKQYKGIIDCVVRIPKEQGVLSFWRGNLANVIRYFPTQALNFAFKDKYKQIFLGGVDKRTQFWRYFAGNLASGGAAGATSLCFVYPLDFARTRLAADVGKAGAEREFKGLGDCLVKIYKSDGIKGLYQGFNVSVQGIIIYRAAYFGIYDTAKGMLPDPKNTHIFISWMIAQSVTAVAGLTSYPFDTVRRRMMMQSGRKGTDIMYTGTLDCWRKIARDEGSKAFFKGAWSNVLRGMGGAFVLVLYDEIKKYT

SEQ ID NO: 9 - DNA, 411 bp, Artificial sequence
  Caspase 3 probe H2B-DEVD
atgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgatgaagtcgatgccaccatg

SEQ ID NO: 10 - protein, 137 aa, Artificial sequence
  Caspase 3 probe H2B-DEVD
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVDEVDATM

SEQ ID NO: 11 - DNA, 414 bp, Artificial sequence
  Caspase 2 probe H2B-VDVAD
atgccagagccagcgaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccatcaccaagtacaccagcgctaaggatccaccggtcgtcgacgtcgccgatgccaccatg

SEQ ID NO: 12 - protein, 138 aa, Artificial sequence
  Caspase 2 probe H2B-VDVAD
MPEPAKSAPAPKKGSKKAVTKAQKKGGKKRKRSRKESYSIYVYKVLKQVHPDTGISSKAMGIMNSFVNDIFERIAGEASRLAHYNKRSTITSREIQTAVRLLLPGELAKHAVSEGTKAITKYTSAKDPPVVDVADATM

SEQ ID NO: 13 - DNA, 177 bp, Artificial sequence
  Caspase 8 probe IETD-cb5RR
gaaggtggaggaggttcaattgaaaccgattcaggaggaggtggatctggaggtggcggatccttcgagccgtccgaaactctgatcactaccgttgaatcgaactcgagttggtggactaactgggttatccctgcgatctctgctctggttgtagcgctgatgtaccggcgttaa

SEQ ID NO: 14 - protein, 58 aa, Artificial sequence
  Caspase 8 probe IETD-cb5RR
EGGGGSIETDSGGGGSGGGGSFEPSETLITTVESNSSWWTNWVIPAISALVVALMYRR